(19) United States
(12) Patent Application Publication
(10) Pub. No.: US 2011/0278343 A1
(43) Pub. Date: Nov. 17, 2011

(54) Clamping of Hybrid Surgical Instrument

(75) Inventors: Bryan D. Knodel, Flagstaff, AZ (US); Jared L. Van Cleave, Menlo Park, CA (US); Jinhoon Park, Palo Alto, CA (US); Michael Murillo, Palo Alto, CA (US); Nathan H. White, Redwood City, CA (US); Philipe R. Manoux, San Francisco, CA (US)

(73) Assignee: CARDICA, INC., Redwood City, CA (US)

(21) Appl. No.: 12/477,302

(22) Filed: Jun. 3, 2009

Related U.S. Application Data
(60) Provisional application No. 61/148,346, filed on Jan. 29, 2009.

(51) Int. Cl. A61B 17/068; A61B 18/14

(52) U.S. Cl. 227/176.1; 606/50

(57) ABSTRACT

One example of a surgical instrument may include a staple holder; staples held by the staple holder; an anvil, where at least one of the staple holder and anvil is movable relative to the other; and at least one electrode located on a surface of the staple holder and/or anvil. Another example of a surgical instrument may include a staple holder; staples held by the staple holder; an anvil, where at least one of the staple holder and the anvil is movable relative to the other between an open configuration and a closed configuration; where an adjustable gap exists between the staple holder and the anvil in the closed configuration; and at least one electrode located on a surface of at least one of the staple holder and/or anvil. An exemplary method may include providing a surgical instrument including an end effector; clamping tissue with the end effector; selecting stapling or electrocautery; and stapling or performing electrocautery accordingly.

This application claims priority to U.S. Provisional Patent Application Ser. No. 61/148,346, filed on Jan. 29, 2009, which is hereby incorporated by reference herein in its entirety.

### FIELD OF THE INVENTION

The invention generally relates to surgical stapling and electrocautery.

### BACKGROUND

An endocutter is a surgical tool that staples and cuts tissue to transect that tissue while leaving the cut ends hemostatic. An endocutter is small enough in diameter for use in minimally invasive surgery, where access to a surgical site is obtained through a trocar, port, or small incision in the body. A linear cutter is a larger version of an endocutter, and is used to transect portions of the gastrointestinal tract. A typical endocutter receives at its distal end a disposable single-use cartridge with several rows of staples, and includes an anvil opposed to the cartridge. The surgeon inserts the endocutter through a trocar or other port or incision in the body, orients the end of the endocutter around the tissue to be transected, and compresses the anvil and cartridge together to clamp the tissue. Then, a row or rows of staples are deployed on either side of the transection line, and a blade is advanced along the transection line to divide the tissue. Electrocautery tools are also known, and have been utilized to both divide and seal tissue.

Currently, coagulation-based devices for dissecting tissue are known and used in surgical procedures. These devices clamp tissue, deliver energy such as RF energy or ultrasound to tissue in order to cause coagulation to heat seal the tissue, then use a knife to cut the tissue. A surgeon may use such a device, for example, to divide the mesenteric tissue in preparation for bowel surgery. However, in the course of such preparation, the surgeon typically encounters blood vessels that are larger than coagulation-based devices can safely dissect. Consequently, the surgeon must then set aside the coagulation-device and switch to an endocutter that can deploy staples. The endocutter, by implanting a plurality of staples, can divide more safely larger blood vessels and other highly vascularized tissue. Switching between tools is time-consuming, expensive, and inconvenient, particularly in minimally-invasive, laparoscopic, or port-access surgical procedures, where the tissue to be dissected can be lost upon withdrawal of the tool such that the surgeon must spend time finding that tissue again upon the insertion of a different tool.

When utilizing an endocutter or other surgical stapler that utilizes an anvil and cartridge or other staple holder, it is generally important to maintain a substantially constant gap between the anvil and the cartridge when the anvil and the cartridge are clamped together. That substantially constant gap is important to proper staple formation. That is, a staple urged outward from the cartridge or other staple holder is designed to encounter a staple pocket or other feature in the anvil at a certain point in its travel. If the staple encounters that staple pocket or other feature in the anvil too soon or too late, the staple may be malformed. For example, if the gap is too large, the staple may not be completely formed. As another example, if the gap is too small, the staple may be crushed. However, when utilizing a bipolar electrocautery tool, it is generally important to move the electrodes as close together as possible. Where such a tool utilizes two jaws, one or more electrodes may be provided on each jaw, where the electrodes on each jaw may collectively form a single pole. Tissue is clamped between the jaws in such a way as to minimize the gap therebetween. In this way, tissue maintains good contact with each jaw to facilitate the flow of current. Air space between the tissue and one or both jaws reduces the effectiveness of the electrocautery tool, and may lead to undesired clinical outcomes. Thus, the requirement for a gap between the anvil and cartridge or other staple holder on the one hand, and the requirement for tight clamping of tissue across a range of thickness for an electrocautery tool on the other, are at odds with one another.

### BRIEF DESCRIPTION OF THE DRAWINGS

FIG. 1 is a perspective view of an exemplary medical device switchable between stapling and coagulation modes, that device including a handle and an end effector.

FIG. 2 is a perspective view of an exemplary end effector of FIG. 1, having a staple holder and an anvil.

FIG. 3 is a top view of the exemplary staple holder of FIG. 2.

FIG. 4 is a side view of one example of an end effector with an exemplary adjustable gap.

FIG. 5 is a side view of the end effector of FIG. 4, in a second configuration.

FIG. 6 is a side view of another example of an end effector with an exemplary adjustable gap.

FIG. 7 is a side view of the end effector of FIG. 6, in a second configuration.

FIG. 8 is a side view of the end effector of FIG. 6, in a third configuration.

FIG. 9 is a side cross-section view of the distal end of an overtube with a two-stage cam surface defined therein.

FIG. 10 is a side view of another example of an end effector with an exemplary adjustable gap.

FIG. 11 is a side view of the end effector of FIG. 10, in a second configuration.

FIG. 12 is a side view of the end effector of FIG. 10, in a third configuration.

FIG. 13 is a front view of another example of an end effector with an exemplary adjustable gap.

FIG. 14 is a front view of the end effector of FIG. 13, in a second configuration.

FIG. 15 is a side view of another example of an end effector with an exemplary adjustable gap.

FIG. 16 is a side view of another example of an end effector with an exemplary adjustable gap

The use of the same reference symbols in different figures indicates similar or identical items.

### DETAILED DESCRIPTION

U.S. patent application Ser. No. 11/851,379, filed Sep. 6, 2007; U.S. patent application Ser. No. 11/956,988, filed Dec. 14, 2007; U.S. patent application Ser. No. 12/263,171, filed Oct. 31, 2008 (the “Endocutter Documents”) are hereby incorporated by reference herein in their entirety. The Endocutter Documents describe a surgical stapler that includes an end effector attached to a shaft, which in turn is attached to a handle. The Endocutter Documents also describe a feeder belt extending into the end effector, where staples extend from and are frangibly connected to the feeder belt.

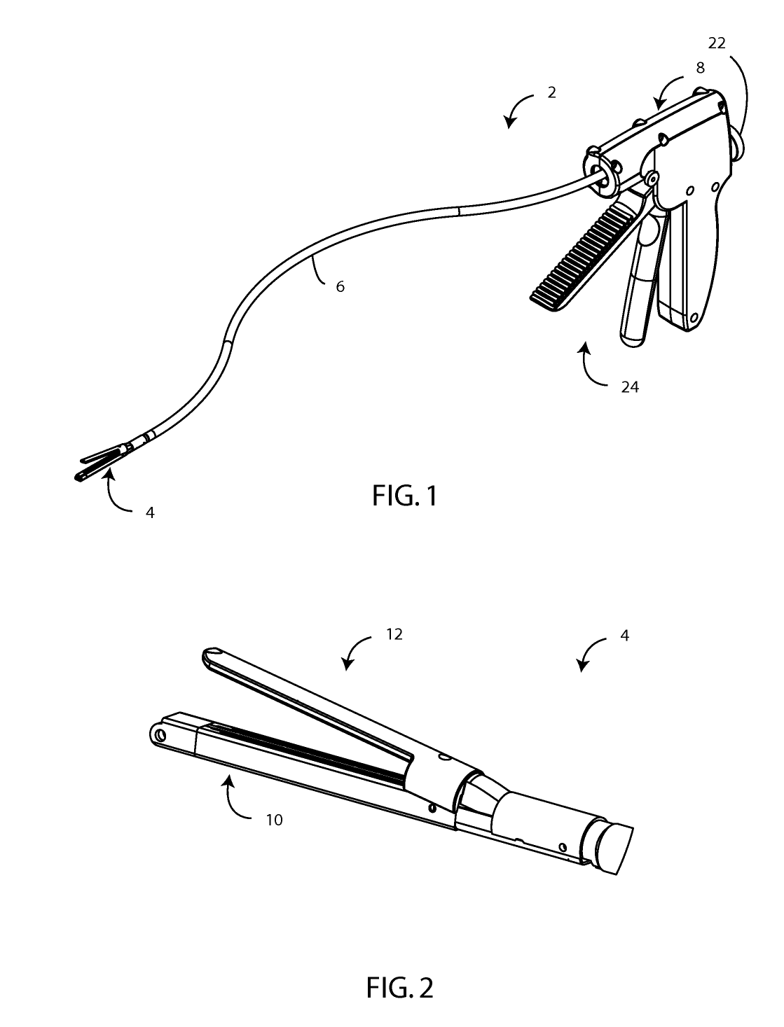

Referring to FIG. 1, a medical device 2 may include an end effector 4 attached to a shaft 6, which in turn is attached to a handle 8. The end effector 4 may be one or more separate components that are connected to the shaft 6, or may be fabricated integrally with the distal end of the shaft 6. The shaft 6 may be rigid or flexible. The end effector 4 and the shaft 6 may be sized to pass through a standard trocar port that may be placed through tissue of a patient. Advantageously, the end effector 4 may be sized to pass through a trocar port having an opening between 5-10 millimeters in diameter. Alternately, the medical device 2 may be used in the course of conventional open surgery, where a trocar port is not used. Alternately, the medical device 2 may be used in the course of minimally-invasive surgery, where access to the surgical site in the patient is gained through a mechanism or structure other than a trocar port, such as the LAP DISC® hand access device of Ethicon Endo-Surgery, Inc., or where access to the surgical site in the patient is gained through an incision or opening in which no port or other mechanism or structure is placed.

The handle 8 may include any mechanism, mechanisms, structure or structures that are suitably configured to actuate the end effector 4. The handle 8 includes at least one source of stored energy for actuating the end effector 4. The source or sources of stored energy may be mechanical (such as a spring), electrical (such as a battery), pneumatic (such as a cylinder of pressurized gas) or any other suitable source of stored energy. One source of stored energy, its regulation, and its use in actuating the end effector 4 may be as described in the U.S. patent application Ser. No. 11/054,265, filed on Feb. 9, 2005, which is hereby incorporated by reference in its entirety herein. The handle 8 may instead, or also, include a connector or connectors suitable for receiving stored energy from an external source, such as a hose connected to a hospital utility source of pressurized gas or of vacuum, or an electrical cord connectable to a power source.

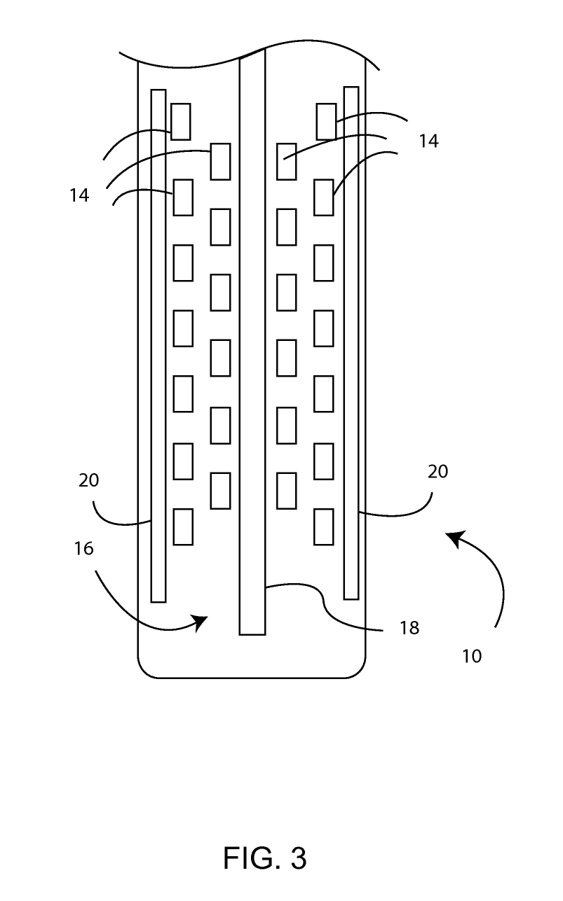

Referring also to FIGS. 2-3, the end effector 4 may include a staple holder 10 and an anvil 12. At least one of the staple holder 10 and anvil 12 may be movable relative to the other in order to compress or clamp tissue therebetween. The end effector 4 may hold a plurality of staples or clips, such as described in the Endocutter Documents. Advantageously, the staple holder 10 is fixed to a remainder of the end effector 4 and/or the shaft 6, and is not detachable therefrom. The staple holder 10 may be fired multiple times without being withdrawn from the patient, such that there is no need to withdraw the end effector 4 from the patient after each deployment of staples or clips in order to replace a staple cartridge or other component. Nevertheless, if desired at least a portion of the staple holder 10 may be detachable from a remainder of the end effector 4 and/or the shaft 6; the end effector 4 may be detachable from the shaft 6; and/or the shaft 6 may be detachable from the handle 8. For example, the staple holder 10 may be configured to hold a detachable cartridge that may be configured to deploy staples multiple times, or only a single time. The end effector 4 may be an endocutter, such as described in the Endocutter Documents, or may be any other suitable device; the end effector 4 is not limited to an endocutter. In such an endocutter, at least one feeder belt extends into the end effector 4, and at least one staple is fixed to and frangibly separable from that feeder belt.

The staples or clips may be deployable through openings 14 in the upper surface 16 of the staple holder 10. The openings 14 may be organized in two or more rows extending generally longitudinally along the upper surface 16 of the staple holder 10. However, the openings 14 may be organized differently. A knife slot 18 may extend along at least part of the upper surface 16 of the staple holder 10, to allow a knife to slide along the slot and divide tissue. The knife slot 18 may be positioned between two separate rows of openings 14. Advantageously, at least two rows of openings 14 may be arranged on each side of the knife slot 18.

At least one coagulation surface 20 is located on the upper surface 16 of the staple holder 10. Each coagulation surface 20 may be oriented generally longitudinally along the upper surface 16 of the staple holder 10. At least one coagulation surface 20 may be generally parallel to at least one row of openings 14 in the upper surface 16 of the staple holder 10, and positioned laterally outward from the openings 14 on one side of the staple holder 10, at or near an edge of the staple holder 10. Alternately, at least one coagulation surface 20 may be generally parallel to at least one row of openings 14 in the upper surface 16 of the staple holder 10, and positioned between the knife slot 18 and a row of openings 14. Alternately, at least one coagulation surface 20 may be oriented differently. Alternately, at least one coagulation surface 20 may be generally U-shaped. The openings 14 may be located within the U-shaped coagulation surface 20, or may be otherwise oriented relative to the U-shaped coagulation surface. Alternately, the entire upper surface 16 of the staple holder 10 may be a coagulation surface 20.

Each coagulation surface 20 is connected to an energy source in the handle 8 in any appropriate manner. For example, one or more wires or waveguides may extend from a coagulation surface 20 through the shaft 6 to the energy source in the handle 8. The energy source connected to each coagulation surface 20 may be different from the energy source used to deploy clips or staples from the staple holder 10. For example, the handle 8 may include a cylinder of pressurized gas used to deploy staples from the staple holder 10, and a connection to an electric power source for actuating the coagulation surfaces 20. The energy source connected to each coagulation surface 20 instead may be the same as the energy source used to deploy clips or staples from the staple holder 10.

Each coagulation surface 20 may be configured in any suitable manner to deliver RF energy, ultrasound, heat, electricity or any other kind of energy to tissue. For example, at least one coagulation surface 20 may be a complete mechanism or other device configured to convert electrical or other energy into a different kind of energy suitable for causing coagulation. Such mechanisms and devices are known in the art. Each coagulation surface 20 may be a pole of a bipolar coagulation device, or each may be unipolar. Alternately, the coagulation surfaces 20 are collectively one pole of a bipolar coagulation device, and the anvil 12 is the other pole. The anvil 12 may include one or more coagulation surfaces as well, arranged on the surface of the anvil 12 in any suitable manner. Alternately, the anvil 12 may include one or more coagulation surfaces instead of the staple holder 10. Alternately, the coagulation surfaces 20 may be collectively monopolar.

Optionally, at least one of the anvil 12 and/or staple holder 10 may be fabricated from ceramic material. At least one coagulation surface 20 may be located directly on such a ceramic anvil 12 and/or staple holder 10, without the need for additional insulation. Alternately, ceramic material may be placed on a surface of the anvil 12 and/or the staple holder 12 underneath a corresponding coagulation surface 20 in order to insulate that coagulation surface 20. Optionally, the ceramic material may be piezoelectric, and one or more feedback wires may be connected to that ceramic material. The feedback wires may be connected to the handle 8 and/or the electrocautery generator. As the anvil 12 and staple holder 10 are moved together to the closed, clamped configuration, the ceramic material generates a piezoelectric voltage that is transmitted through the feedback wires. In this way, the clamping force exerted on the anvil 12 and staple holder 10 may be adjusted based on feedback received through the feedback wires.

The end effector 4 may be configured to deploy staples from the staple holder 10 or to apply energy to the coagulation surfaces 20, based on the selection of the user. The handle 8 may include a switch 22 that allows the user to switch between stapling mode and coagulation mode. Alternately, the switch 22 may be located elsewhere than the handle 8, and be connected to the medical device 2. For example, the switch 22 may be a foot pedal connected electrically to the handle 8. Such a switch 22 may be mechanical, electrical, a combination thereof, or a different kind of switch. The switch 22 may cause the medical device 2 to switch between modes in any suitable manner. For example, motion of the switch 22 may physically lock out the staples from firing, such as by moving a tab into engagement with a valve connected to a cylinder of gas that powers the stapling mechanism of the end effector, and at substantially the same time switch on or otherwise complete a circuit to allow actuation of the coagulation surfaces 20. By switching between modes, a single trigger 24 could be used to actuate the end effector in each mode. Alternately, the switch 22 may be omitted, and the handle 8 may include two or more triggers 24 or other actuators, such that the user actuates one trigger 24 to deploy staples and a second trigger 24 to actuate the coagulation surfaces. Alternately, the handle 8 may include any other features that allow the user to selectively deploy staples and coagulate tissue. Alternately, the end effector 4 may be configured to deploy staples from the staple holder 10 and apply energy to the coagulation surfaces 20 at the same time, either at the selection of the user, or as the only mode of operation of the medical device 2. The source of energy applied to coagulation surfaces 20 may be a standard electrocautery generator.

Clamping

Before the end effector 4 is actuated to staple or cauterize tissue, the end effector 4 is moved to a closed, clamped configuration. Where stapling is desired, it is desirable that a substantially constant, known gap is present between the anvil 12 and staple holder 10 when the end effector 4 is in the clamped configuration. Where electrocautery is desired, it is desirable that the gap between the anvil 12 and the staple holder 10 is as small as possible when the end effector 4 is in the clamped configuration. As a result, the end effector 4 may be controllable to clamp tissue between the anvil 12 and the staple holder 10 in a first manner in order to maintain a substantially constant gap therebetween, and in a second manner in order to minimize the gap therebetween. That is, there is an adjustable gap between the anvil 12 in the staple holder 10.

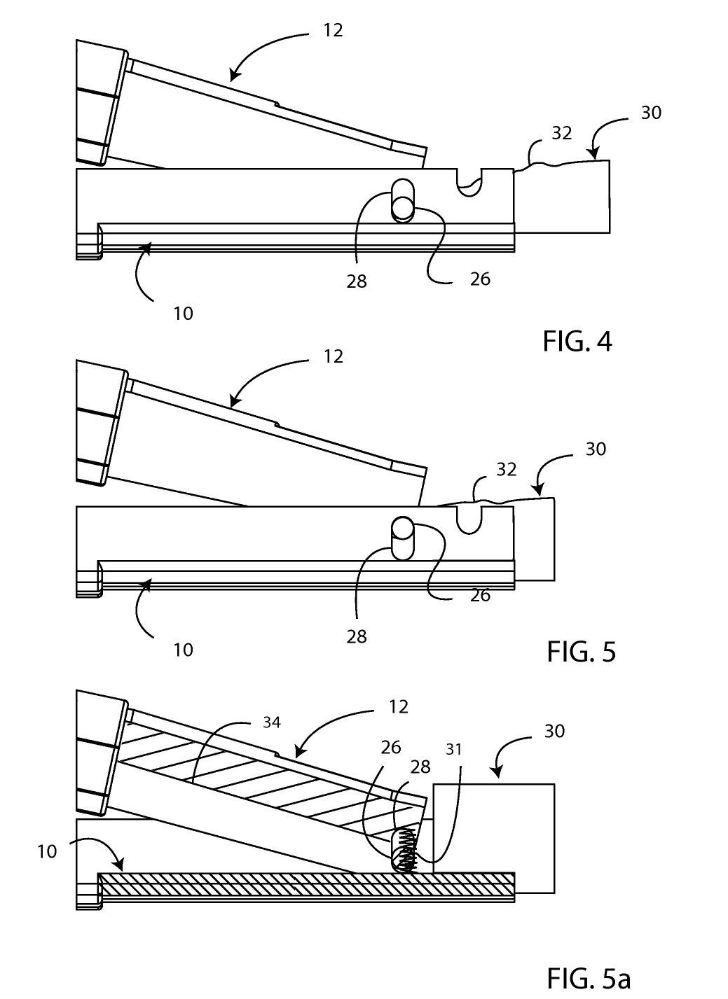

Referring to FIG. 4, as one example, a pin 26 may be located on the anvil 12, at the proximal end thereof, at or near the bottom surface of the anvil 12. The use of the term “bottom” refers to the orientation of the anvil 12 on the page for the convenience of the reader, and does not limit the orientation of the anvil 12 in use. The pin 26 may be generally cylindrical, and extend laterally outward from the anvil 12. Advantageously, two pins 26 are utilized, one on either side of the anvil 12, where Such pins 26 are arranged generally symmetrically. Each pin 26 extends into a slot 28 in the staple holder 10. Each slot 28 may be generally vertically oriented, and maybe located at or near the proximal end of the staple holder 10. Each slot 28 constrains the motion of the corresponding pin 26. A stepped adjustment bar 30 may be slidable along the staple holder 10, between the staple holder 10 and the anvil 12. The adjustment bar 30 may slide within a corresponding slot in the staple holder 10 and another corresponding slot 34 in the anvil 12. The upper surface 32 of the adjustment bar 30 may include a plurality of discrete steps. Alternately, the upper surface 32 of the adjustment bar 30 may be angled, curved, continuous, or otherwise shaped. Referring also to FIG. 5, as the adjustment bar 30 is moved distally, the upper surface 32 of the adjustment bar 30 may contact the proximal end of the slot 34, urging the proximal end of the slot 34 upward and thereby urging the anvil 12 upward. As the anvil 12 moves upward, each pin 26 moves upward in the corresponding slot 28. As each pin 26 moves upward, the pivot point between the anvil 12 and the staple holder 10 moves upward. Consequently, as the pivot point moves upward, the gap between the anvil 12 and the staple holder 10 in the clamped configuration increases. Thus, the adjustment bar 30 may be in a first position relative to the anvil 12 for stapling, such that a comparatively large gap between the anvil 12 and staple holder 10 is provided, and the adjustment bar 30 may be moved proximally to decrease that gap for electrocautery. Advantageously, the proximal end of the anvil 12 may be biased downward, such as by a spring, such that the adjustment bar 30 counteracts that bias when it moves the anvil 12 upward.

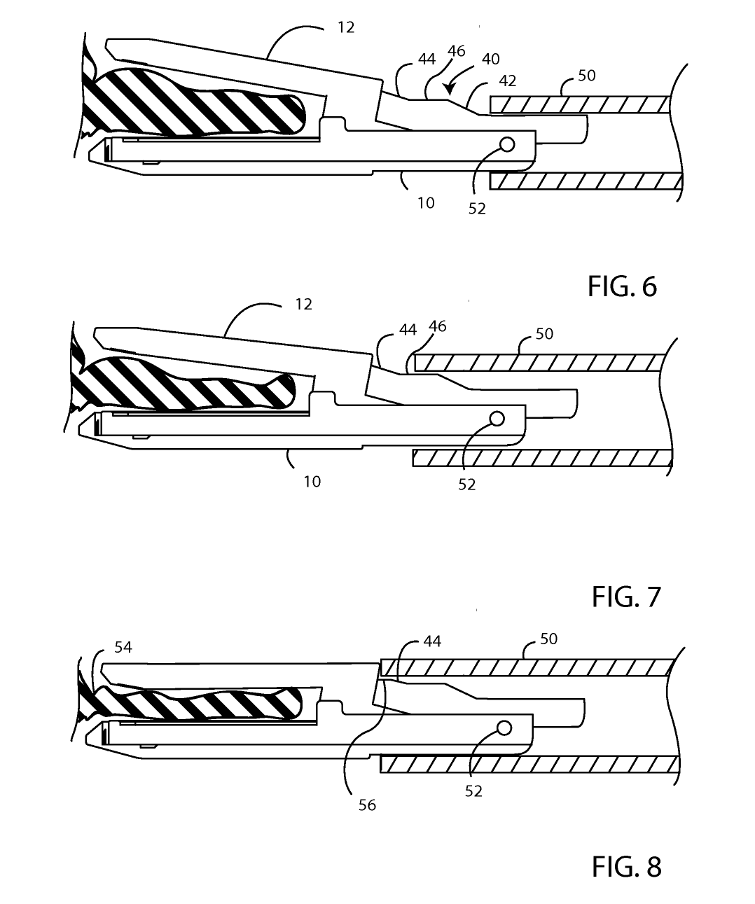

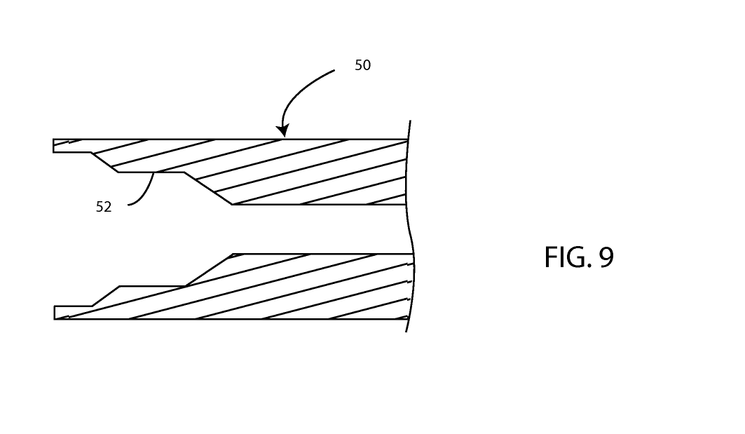

As another example of gap adjustment, referring also to FIG. 6, the proximal end of the anvil 12 may include a two-stage cam surface 40. Such a cam surface includes two ramps 42, 44 that extend upward in the distal direction. The ramps 42, 44 maybe separated by a flat area 46 or other spacer. The shaft 6 may include an overtube 50 at its distal end, or slidable over or within its distal end. In the initial configuration, the proximal end of the anvil 12 and the staple holder 10 each may extend into the lumen of the overtube 50. The anvil 12 and staple holder 10 may be rotatably connected at a pivot point 52. As the overtube 50 is advanced distally, the distal end of the overtube 50 encounters the first ramp 42, which is located proximal to the second ramp 44. Referring also to FIG. 7, as the distal end of the overtube 50 presses against the first ramp 42, the anvil 12 pivots downwards about the pivot point 52 toward the staple holder 10. The overtube 50 advances until the flat area 46 enters the lumen of the overtube 50. At this point, the anvil 12 and the staple holder 10 are spaced apart a first distance suitable for stapling. The gap between the anvil 12 and the staple holder 10 is known it is suitable for staple deployment. Referring also to FIG. 7, the overtube 50 may be advanced distally further until the distal end of the overtube encounters the second ramp 44. As the distal end of the overtube 50 presses against the second ramp 44, the anvil 12 pivots downwards about the pivot point 52 toward the staple holder 10. The overtube 50 may be advanced until tissue 54 located between the anvil 12 and the staple holder 10 prevents further clamping, or until the overtube 50 reaches a flat area 56, a stop on the anvil 12, or the end of its travel. At this point, the anvil 12 and the staple holder 10 are spaced apart a second distance suitable for cauterization, for the second distance is smaller than the first distance. Alternately, the staple holder 10 may also, or instead, include a two-stage cam surface 40. Alternately, referring also to FIG. 9, the overtube 50 may also, or instead, include a two-stage cam surface 52 defined in the distal end of the lumen thereof. Such a cam surface 52 to be the inverse of, and act in substantially same manner as, the two-stage cam surface 40 described above. The overtube 50 may include the two-stage cam surface 52 instead of, or in addition to, the two-stage cam surface 40 of the anvil 12 and/or staple holder 10.

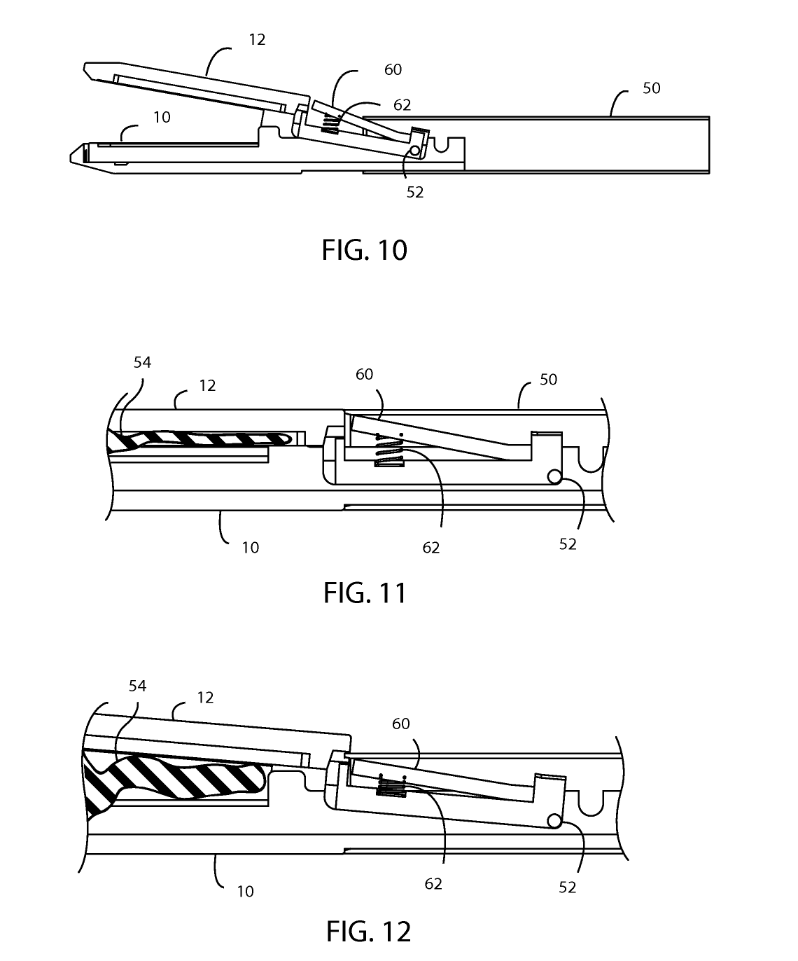

As another example of gap adjustment, referring also to FIG. 10, the proximal end of the anvil 12 may include an upwardly-biased cam arm 60. The cam arm 60 may be oriented upward in the distal direction. The distal end of the cam arm 60 may be biased upward in any suitable manner. For example, a spring 62 may be positioned between the underside of the cam arm 60 and the anvil 12. In the initial configuration, the proximal end of the anvil 12 and the staple holder 10 each may extend into the lumen of the overtube 50. The anvil 12 and staple holder 10 may be rotatably connected at a pivot point 52, which initially maybe located within the lumen of the overtube 50. As the overtube 50 is advanced distally, the distal end of the overtube 50 pushes against the angled surface of the cam arm 60. Referring to FIG. 11, where the tissue 54 to be clamped between the anvil 12 and the staple holder 10 is sufficiently thin, the force required to clamp that tissue 54 is less than the force with which the spring 62 biases the distal end of the cam arm 60 upward. Consequently, the force exerted by the distal end of the overtube 50 against the cam arm 60 substantially does not deflect the cam arm 60, and instead rotates the anvil 12 downwards to a fully-clamped position relative to the staple holder 10. In the fully-clamped position, the gap between the anvil 12 and the staple holder 10 may be a first distance, which may be sized to facilitate stapling of the tissue 54. Optionally, tissue 54 may be cauterized when the gap between the anvil 12 and the staple holder 10 is at the first distance. Referring to FIG. 12, where the tissue 54 to be clamped between the anvil 12 and the staple holder 10 is thicker, the force required to clamp that tissue 54 is greater than the force with which the spring 62 biases the distal end of the cam arm 60 upward. Consequently, the force exerted by the distal end of the overtube 50 against the cam arm 60 deflects the cam arm 60 downward. As a result, the anvil 12 does not close completely, because at least a portion of the force exerted by the overtube 50 against the cam arm 60 deflects the cam arm 60 downward instead of deflecting the anvil 12 downward. That is, the cam arm 60 limits the total amount of force that can be applied to tissue 54, thereby placing an upper limit on the maximum clamping force. The tissue 54 may then be stapled or cauterized, at the discretion of the user.

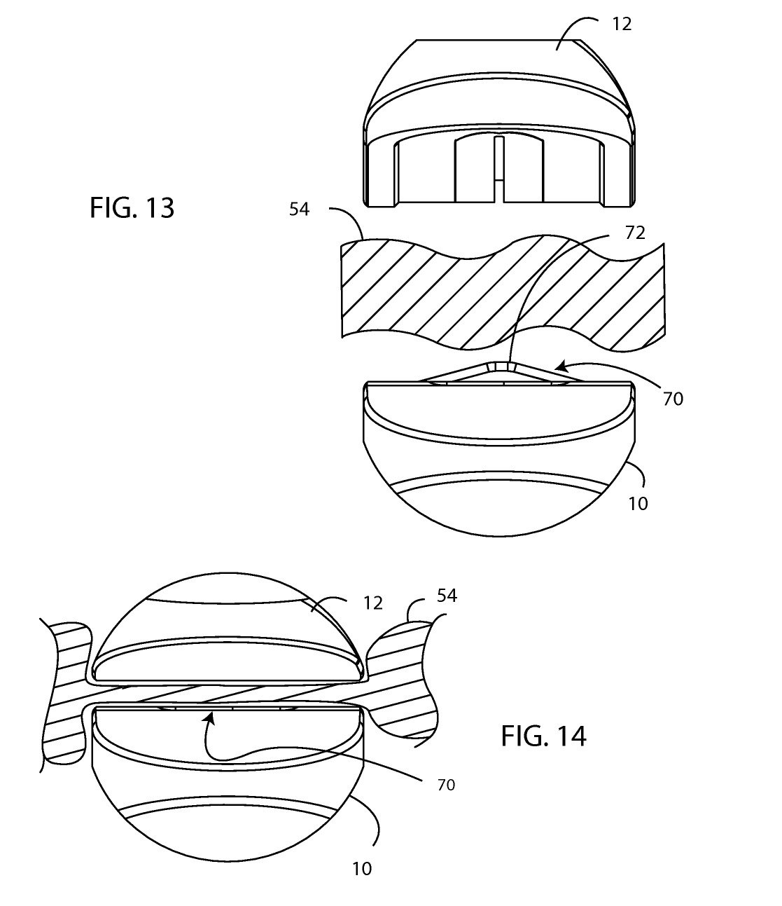

As another example, referring also to FIG. 13, the staple holder 10 may include a flexible convex surface 70 facing the anvil 12. Alternately, the flexible convex surface 70 may be positioned on the anvil 12 facing the staple holder 10. The flexible convex surface 70 may be a leaf spring or any other suitable mechanism. The flexible convex surface 70 may be shaped in any suitable manner, with a peak 72 that may be located laterally substantially at the center of the flexible convex surface 70 and that may extend longitudinally along the length of the flexible convex surface 70. However, the peak 72 may be configured in any other suitable manner. The flexible convex surface 70 is configured to retain its shape until a particular force is applied to it, at which time the flexible convex surface 70 deflects and flattens. As the anvil 12 and the staple holder 10 are moved closer together, the peak 72 of the flexible convex surface 70 may act as a stop, preventing further motion together of the anvil 12 and the staple holder 10. At this point, the gap between the anvil 12 and the staple holder 10 may be a first distance that is advantageously sized for the deployment of staples into tissue 54. Referring also to FIG. 14, as greater force is applied to the anvil 12 and/or staple holder 10 to urge them together, that force deflects and flattens the flexible convex surface 70, overcoming the resistance previously given by the peak 72. Thus, as the peak 72 collapses, the anvil 12 and staple holder 10 can move closer together and compress the tissue 54 to a greater degree. At this point, the gap between the anvil 12 and the staple holder 10 may be at a second distance advantageously sized for cauterization of the tissue 54. Further, the convex surface 70 provides for desiccation of the clamped tissue 54 by “squeegee” action resulting from compression between the convex surface 70 and the opposed surface of the staple holder 10 or anvil 12. This may be particularly useful for the tissue cauterization mode.

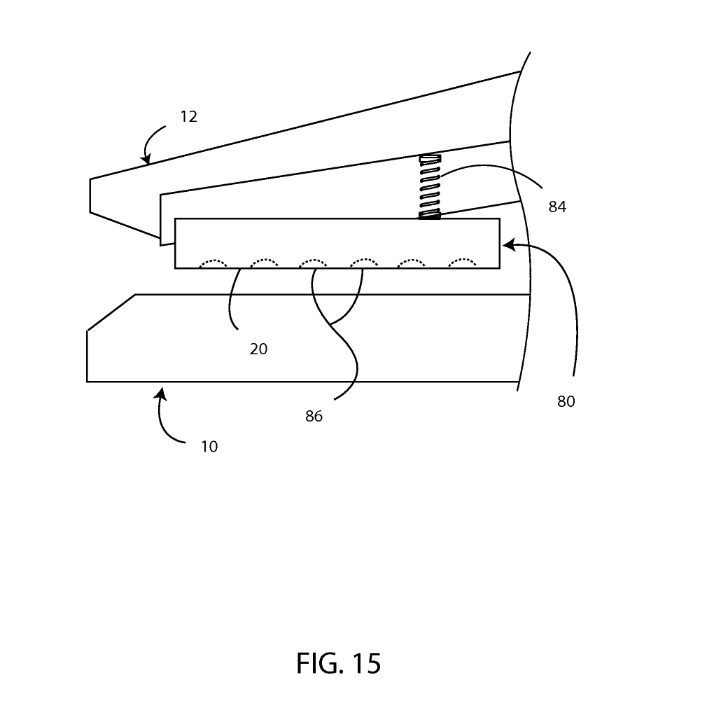

As another example, referring also to FIG. 15, at least one coagulation surface 20 may float relative to the anvil 12 and/or staple holder 10. That coagulation surface 20 may be located on a separate electrode carrier 80, or may not need or utilize a separate electrode carrier 80. The electrode carrier 80 may be pivotally connected to the anvil 12 at a pivot point 82 located near the distal end of the electrode carrier 80, and that also may be located near the distal end of the anvil 12. The proximal end of the electrode carrier 80 may be biased downward by a spring 84 or by any other suitable mechanism or structure. That is, the electrode carrier 80 and corresponding coagulation surface 20 may be biased away from the anvil 12. The spring 84 exerts a known amount of force on the electrode carrier 80 in the direction toward the staple holder 10, and thereby holds the lower surface of the electrode carrier 80 and thus the coagulation surface or surfaces 20 generally parallel to the upper surface of the staple holder 10. Consequently, where tissue below a certain thickness is clamped between the anvil 12 and staple holder 10, the electrode carrier 80 does not deflect upward, such that a known gap is present between the lower surface of the electrode carrier 80 and the upper surface of the staple holder 10. The electrode carrier 80 may be configured with staple forming pockets 86 or other structures against which staples can be ejected from the staple holder 10 and deformed. Where tissue above a certain thickness is clamped between the anvil 12 and staple holder 10, the clamping force required to clamp that tissue causes compression of the spring 84. In this way, the lower surface of the electrode carrier 80 maintains suitable contact against tissue in a variety of tissue thicknesses.

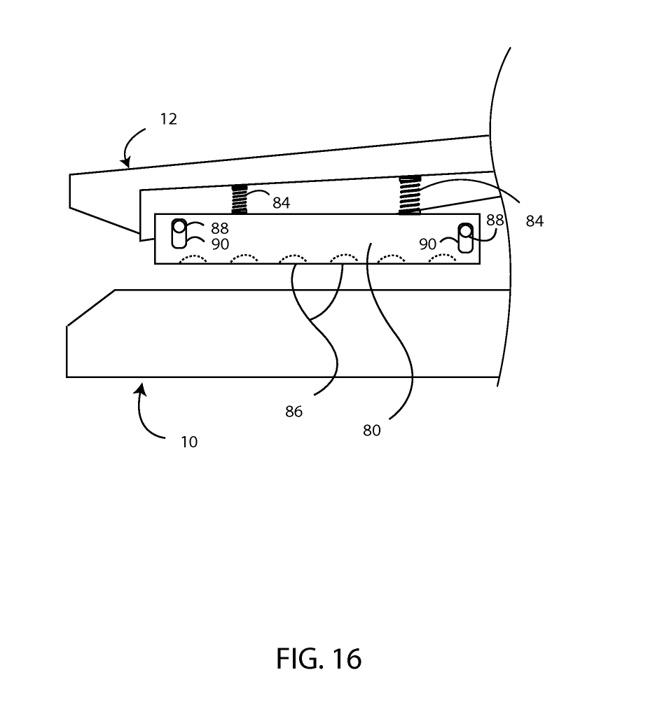

As another example, referring also to FIG. 16 at least one coagulation surface 20 may float relative to the anvil 12 and/or staple holder 10 in a different manner than the coagulation surface 20 of FIG. 15. An electrode carrier 80 may be connected to the anvil 12 via one or more pins 88 extending from the anvil 12, where each pin 88 is received in a corresponding slot 90 in the electrode carrier 80. The electrode carrier 80 may be biased downward by one or more springs 84 or by any other suitable mechanism or structure. That is, the electrode carrier 80 and corresponding coagulation surface 20 may be biased away from the anvil 12. The spring or springs 84 exert a known amount of force on the electrode carrier 80 in the direction toward the staple holder 10. The electrode carrier 80 is slidable along the slots 90, allowing the electrode carrier numeral 80 to float upward and downward relative to the staple holder 10. One of the slots 90 may be shorter than one or more of the other slots 90, allowing for rotation of the electrode carrier 80 about an axis formed by or generally parallel to at least one pin 88. Because the proximal and distal ends of the electrode carrier 80 can rock, the electrode carrier 80 can maintain contact with tissue across its length. Further, the electrode carrier 80 may be biased away from the anvil 12 in such a way that the coagulation surface or surfaces 20 on the lower surface of the electrode carrier 80 are generally parallel to the upper surface of the staple holder 10 when thinner tissue is clamped between the staple holder 10 and the anvil 12.

Operation

The operation of the medical device 2 is described in an exemplary manner, in which the end effector 4 is an endocutter. However, the medical device 2 is not limited to being an endocutter, and instead may be any medical device that is used to dissect tissue and/or seal tissue. The end effector 4 is positioned by the user at a surgical site. As one example, a surgical site is located on a blood vessel which is to be transected. At least the distal end of the anvil 12 is initially spaced apart from the staple holder 10, such that the end effector 4 is open. The end effector 4 is advanced over the blood vessel to be transected, until the entire diameter of the blood vessel is located between the anvil 12 and the staple holder 10. Advantageously, the blood vessel is substantially at a right angle to the anvil 12 and the staple holder 10. However, the blood vessel may be oriented at any other suitable angle relative to the anvil 12 and the staple holder 10. The end effector 4 is then closed, by moving the anvil 12 closer to the staple holder 10, such that the blood vessel is compressed between the anvil 12 and the staple holder 10. Such closure of the end effector 4 may be accomplished in any standard manner or any other suitable manner. As one example, a tube may be advanced distally over the outer surface of both the anvil 12 and the staple holder 10, compressing the anvil 12 and the staple holder 10 together. Alternately, the anvil 12 may be substantially fixed relative to a remainder of the end effector 4 and/or the shaft 6, and the staple holder 10 may be moved closer to the anvil 12 in order to close the end effector 4. Alternately, both the anvil 12 and the staple holder 10 are movable toward one another in order to close the end effector 4. Closure of the end effector 4 may be performed by actuating one or more controls on the handle 8, and/or by releasing energy stored in the handle 8. After the end effector 4 has been closed, the tissue to be treated is held securely by, and affirmatively controlled by, the end effector 4.

Prior to or after closure of the end effector 4, the user may select whether to staple the blood vessel at the surgical site, or coagulate it. The user may also select how tissue is clamped. That is, adjustment of the gap between the anvil 12 in the staple holder 10 in the clamped configuration may be selected. This selection may take place before, during, or after selection of stapling or coagulation. Alternately, tissue clamping is performed automatically in a certain manner based on selection of stapling or coagulation. Clamping of tissue may be performed as set forth above, in any suitable manner. The user may select stapling, such as with the switch 22. The user then actuates one or more controls on the handle 8 to actuate the end effector 4. As a result, staples are deployed through the openings 14, and a knife slides along the knife slot 18, to transect the blood vessel, such as described in the Endocutter Documents. The end effector 4 is then unclamped or otherwise opened to release the transected blood vessel. The user may then move the end effector 4 to another blood vessel or other tissue, and clamp or otherwise close the end effector 4 again without removing the end effector 4 from the patient, such as through a trocar or other port through the outer surface of the patient's body. If the blood vessel is suitable for stapling, that tissue may be stapled and transected. Alternately, the user may select coagulation for transecting the tissue. The user may do so by moving the switch 22, or actuating the trigger 24 associated with coagulation. The energy source in the handle 8 then transmits energy to the coagulation surfaces 20, causing the tissue of the blood vessel that is in contact with the coagulation surfaces to coagulate and seal. A knife slides along the knife slot 18 to transect the blood vessel. The end effector 4 is then opened to release the transected blood vessel. The medical device 2 can thus be used repeatedly in the patient to transect tissue at multiple surgical sites, both with staples and with coagulation. Optionally, stapling and coagulation (such as by electrocautery) are mutually exclusive, meaning that the user may select one but not both at the same time. The user may continue in this manner until the staples are exhausted or until the treatment of tissue in the body is complete. The use of a multiple-fire stapling apparatus such as described in the Endocutter Documents facilitates the repeated use of the medical device 2 to treat tissue of different sizes, thicknesses and/or types within a patient. For example, after firing a group of staples, a plurality of fresh staples may be advanced into the staple holder 10 from the shaft 6 and/or from a remainder of the medical device 2. In this way, the time and material needed to treat tissue within the patient may be reduced compared to conventional medical devices.

The terms “upper,” “lower,” “upward,” “downward,” “up,” “down,” “below,” “above,” “vertical,” and the like are used solely for convenience in this document; such terms refer to directions on the printed page and do not limit the orientation of the surgical stapler in use. While the invention has been described in detail, it will be apparent to one skilled in the art that various changes and modifications can be made and equivalents employed, without departing from the present invention. It is to be understood that the invention is not limited to the details of construction, the arrangements of components, and/or the method set forth in the above description or illustrated in the drawings. Statements in the abstract of this document, and any summary statements in this document, are merely exemplary; they are not, and cannot be interpreted as, limiting the scope of the claims. Further, the figures are merely exemplary and not limiting. Topical headings and subheadings are for the convenience of the reader only. They should not and cannot be construed to have any substantive significance, meaning or interpretation, and should not and cannot be deemed to indicate that all of the information relating to any particular topic is to be found under or limited to any particular heading or subheading. Therefore, the invention is not to be restricted or limited except in accordance with the following claims and their legal equivalents.

What is claimed is:

1. Surgical apparatus, comprising:
a staple holder;
a plurality of staples held by said staple holder;
an anvil, wherein at least one of said staple holder and said anvil is movable relative to the other; and
at least one electrode located on a surface of at least one of said staple holder and said anvil.

2. The surgical apparatus of claim 1, incorporating by reference all of the elements of that claim; further comprising at least one feeder belt, wherein at least part of at least one said feeder belt extends into said staple holder; wherein at least one of said staples is fixed to and frangibly separable from a corresponding said feeder belt.

3. The surgical apparatus of claim 1, incorporating by reference all of the elements of that claim; wherein at least one said electrode is located on said staple holder and on said anvil, said at least one electrode on said anvil facing said at least one electrode on said staple holder; wherein said at least one electrode on said staple holder defines a first pole and said at least one electrode on said anvil defines a second pole.

4. The surgical apparatus of claim 1, incorporating by reference all of the elements of that claim; wherein said staple holder includes an upper surface through which a plurality of apertures are defined, whereby said staples exit said staple holder through said apertures; wherein at least one said electrode is positioned on said upper surface laterally outward from said apertures.

5. The surgical apparatus of claim 1, incorporating by reference all of the elements of that claim; wherein at least one said electrode is generally U-shaped.

6. The surgical apparatus of claim 1, incorporating by reference all of the elements of that claim; wherein at least part of at least one of said anvil and said staple holder is ceramic, wherein at least one said electrode is positioned on and insulated by said ceramic.

7. Surgical apparatus, comprising:
a staple holder;
a plurality of staples held by said staple holder;
an anvil, wherein at least one of said staple holder and said anvil is movable relative to the other between an open configuration and a closed configuration; and wherein an adjustable gap exists between said staple holder and said anvil in said closed configuration; and
at least one electrode located on a surface of at least one of said staple holder and said anvil.

8. The surgical apparatus of claim 7, incorporating by reference all of the elements of that claim; further comprising at least one feeder belt, wherein at least part of at least one said feeder belt extends into said staple holder; wherein at least one of said staples is fixed to and frangibly separable from a corresponding said feeder belt.

9. The surgical apparatus of claim 7, incorporating by reference all of the elements of that claim; wherein said anvil is rotatable relative to said staple holder about a pin, and wherein said staple holder includes a generally vertically-oriented slot that receives said pin; wherein said anvil is biased toward said open configuration; further comprising a stepped adjustment bar slidable between said staple holder and said anvil to selectively urge said anvil away from said staple holder and thereby move said pin upward in said slot.

10. The surgical apparatus of claim 7, incorporating by reference all of the elements of that claim; wherein the proximal end of at least one of said anvil and said staple holder includes a two-stage cam surface; further comprising an overtube slidable relative to said anvil and said staple holder to engage successively each said cam surface.

11. The surgical apparatus of claim 7, incorporating by reference all of the elements of that claim; further comprising an overtube having a lumen therein, said overtube slidable relative to said anvil and said staple holder, wherein the distal end of said lumen of said overtube includes a two-stage cam surface slidable to engage the proximal end at least one of said anvil and said staple holder.

12. The surgical apparatus of claim 7, incorporating by reference all of the elements of that claim; further comprising an upwardly-biased cam arm connected to the proximal end of said anvil and an overtube slidable relative to said anvil to engage said cam arm.

13. The surgical apparatus of claim 7, incorporating by reference all of the elements of that claim; wherein at least one said electrode is attached to and biased away from said anvil.

14. The surgical apparatus of claim 13, incorporating by reference all of the elements of that claim; wherein one end of at least one said electrode is pivotally connected to said anvil.

15. The surgical apparatus of claim 7, incorporating by reference all of the elements of that claim; wherein at least one of said anvil and said staple holder includes a flexible convex surface oriented toward the other of said anvil and said staple holder, wherein said flexible convex surface deflects and flattens upon application of a threshold force thereto.

16. The surgical apparatus of claim 7, incorporating by reference all of the elements of that claim; wherein at least part of at least one of said anvil and said staple holder is ceramic, wherein at least one said electrode is positioned on and insulated by said ceramic.

17. The surgical apparatus of claim 16, incorporating by reference all of the elements of that claim; further comprising a feedback wire connected to said ceramic, wherein said ceramic generates a piezoelectric current in said closed configuration based on stress experienced by said anvil, and wherein said current is transmitted along said feedback wire.

18. A surgical method of treating tissue within the body of a patient, comprising:
providing a surgical instrument including an end effector;
clamping tissue with said end effector;
selecting an operating mode from the group consisting of stapling and electrocautery; and
actuating said surgical instrument according to said selecting.

19. The surgical apparatus of claim 18, incorporating by reference all of the elements of that claim; wherein said end effector includes an anvil and a staple holder, at least one of which is movable relative to the other; further comprising performing said selecting before said clamping; wherein said clamping adjusts a gap between said staple holder and said anvil based on said selecting.

20. The surgical apparatus of claim 18, incorporating by reference all of the elements of that claim; further comprising unclamping said end effector, and repeating said clamping and said selecting without removing said surgical instrument from the patient.

21. The surgical apparatus of claim 18, incorporating by reference all of the elements of that claim; wherein said stapling and said electrocautery are mutually exclusive.

22. The surgical apparatus of claim 18, incorporating by reference all of the elements of that claim; further comprising, after said stapling, advancing a plurality of staples into said end effector from a remainder of said surgical instrument.
